(19) United States
(12) Patent Application Publication
(10) Pub. No.: US 2022/0160325 A1
(43) Pub. Date: May 26, 2022

(54) METHOD OF DETERMINING RESPIRATORY STATES AND PATTERNS FROM TRACHEAL SOUND ANALYSIS

(71) Applicant: RTM Vital Signs LLC, Philadelphia, PA (US)

(72) Inventors: Moeness G. Amin, Berwyn, PA (US); InduPriya Eedara, Wayne, PA (US)

(21) Appl. No.: 17/102,545

(22) Filed: Nov. 24, 2020

(51) Int. Cl. A61B 7/00; A61B 5/00; A61B 5/091

(52) U.S. Cl. CPC A61B 7/003; A61B 5/7264; A61B 5/6822; A61B 5/091; A61B 5/6833; A61B 5/7239

(57) ABSTRACT

A method of determining respiratory states, comprising measuring an unfiltered sound waveform emanating from an airflow through a mammalian trachea and applying time-averages to each of a plurality of respiratory phases of the unfiltered sound waveform to create normalized and unnormalized autocorrelation function (ACF) curves. Determining from the normalized and unnormalized ACF curves at least one feature from a first group of features consisting of (a) a first minimum value of the normalized ACF curve; (b) a second maximum value of the normalized ACF curve; (c) a value of the unnormalized ACF curve at zero lag; (d) variance after the normalized ACF curve second maximum value; (e) slope after the normalized ACF curve second maximum value; and (f) sum of the squares of the difference between successive normalized ACF curve maximum and minimum values. Applying a classifier to the at least one feature from the group of features.

### CROSS-REFERENCE TO RELATED APPLICATION

This application is related to and claims priority to U.S. Provisional Patent Application Ser. No. 62/939,864, filed Nov. 25, 2019, entitled METHOD OF DETERMINING RESPIRATORY STATES AND PATTERNS FROM TRACHEAL SOUND ANALYSIS, the entirety of which is incorporated herein by reference.

### FIELD

This disclosure relates to a method and system for determining respiratory states and patterns from tracheal sound analysis.

### BACKGROUND

Respiratory sound analysis provides valuable information about airway structure and respiratory disorders. They are a measure of the body surface vibrations set into motion by pressure fluctuations. These pressure variations are transmitted through the inner surface of the trachea from turbulent airflow in the airways. The vibrations are determined by the magnitude and frequency content of the pressure and by the mass, elastance and resistance of the tracheal wall and surrounding soft tissue.

Regarding heart sounds, the signals acquired at the suprasternal notch are intrinsically different to those observed at the surface of the chest. Signals measured at the chest have travelled a short distance propagating from the heart, through lung tissue and finally through muscle and bone. Signals measured at the suprasternal notch have travelled a greater distance from the heart and principally propagated along the arterial wall of the carotid artery. As a result, the heart sound signals are of similar timing characteristics but of significantly lower bandwidth.

The use of a single sensor to measure the combined acoustic sounds of two activities, namely heartbeats and respiratory sounds, however, cause them to mutually interfere with each other. In essence, one challenge in examining the respiratory condition and classifying its normality or abnormality is the presence of heartbeats in data measurements. Heartbeats have their own acoustic power and signatures, and if not removed from the tracheal sound data, breathing diagnosis based on tracheal sounds can prove difficult and be sometimes ineffective. There comes the challenge of how to separate the two sounds in order to evaluate each respective function separately. Despite its almost periodic signature and harmonic structure, effective removal of heartbeat sound signal components from the tracheal sound data without compromising or altering the respiratory sound component is still an open problem.

### SUMMARY

Some embodiments advantageously provide a method and system for determining respiratory states and patterns from tracheal sound analysis. In one aspect, a method of determining respiratory states includes measuring an unfiltered sound waveform emanating from an airflow through a mammalian trachea for a predetermined time period. Time-averages are applied to each of a plurality of respiratory phases of the unfiltered sound waveform to create normalized and unnormalized autocorrelation function (ACF) curves. At least one feature from a first group of features consisting of: (a) a first minimum value of the normalized ACF curve; (b) a second maximum value of the normalized ACF curve; (c) a value of the unnormalized ACF curve at zero lag; (d) variance after the normalized ACF curve second maximum value; (e) slope after the normalized ACF curve second maximum value; and (f) sum of the squares of the difference between successive normalized ACF curve maximum and minimum values is determined. Applying a classifier to the at least one feature from the group of features. A respiratory state of a plurality of respiratory states is determined based at least in part on the classification of the at least one features from the first group of features.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 is a front view of an exemplary acoustic device and controller constructed in accordance with the principles of the present application;

FIG. 2A is a graph of unfiltered sound data as a function of amplitude over time for regular breathing;

FIG. 2B. is a graph of filtered sound data as a function of amplitude over time for regular breathing;

FIG. 3A is a graph of unfiltered sound data as a function of amplitude over time for deep breathing;

FIG. 3B. is a graph of filtered sound data as a function of amplitude over time for deep breathing;

FIG. 4A is a graph of unfiltered sound data as a function of amplitude over time for shallow breathing;

FIG. 4B. is a graph of filtered sound data as a function of amplitude over time for shallow breathing;

FIG. 5 is a flow chart of a method of determining a respiratory state of the present application;

FIG. 6A is a graph of an exemplary ACF at different lags of the unfiltered sound data shown in FIG. 3A for the inhale phase of breathing;

FIG. 6B is a graph of an exemplary ACF at different lags of the unfiltered sound data shown in FIG. 3A for the exhale phase of breathing;

FIG. 7A is a graph of an exemplary ACF at different lags of the unfiltered sound data shown in FIG. 2A for the inhale phase of breathing;

FIG. 7B is a graph of an exemplary ACF at different lags of the unfiltered sound data shown in FIG. 2A for the exhale phase of breathing;

FIG. 8A is a graph of an exemplary ACF at different lags of the unfiltered sound data shown in FIG. 4A for the inhale phase of breathing; and

FIG. 8B is a graph of an exemplary ACF at different lags of the unfiltered sound data shown in FIG. 4A for the exhale phase of breathing.

### DETAILED DESCRIPTION

Before describing in detail exemplary embodiments, it is noted that the embodiments reside primarily in combinations of apparatus components and processing steps related to a system and method of determining respiratory states and patterns from tracheal sound analysis. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

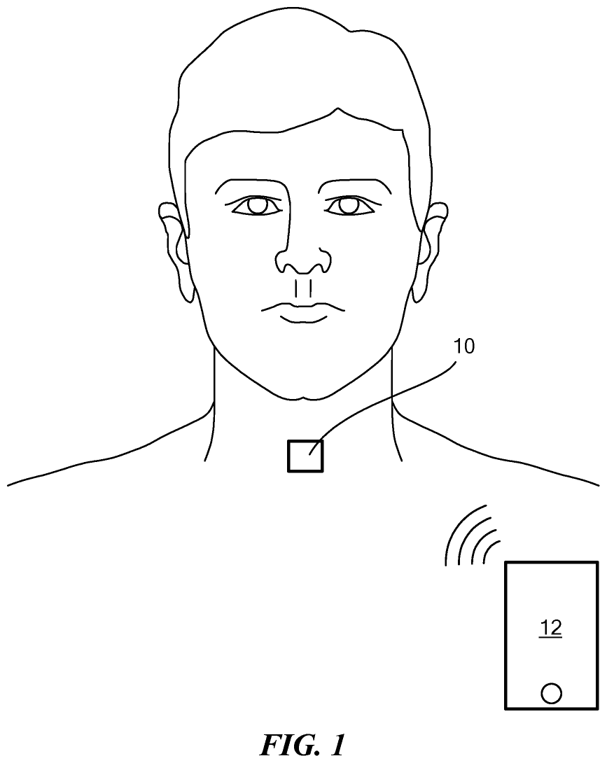

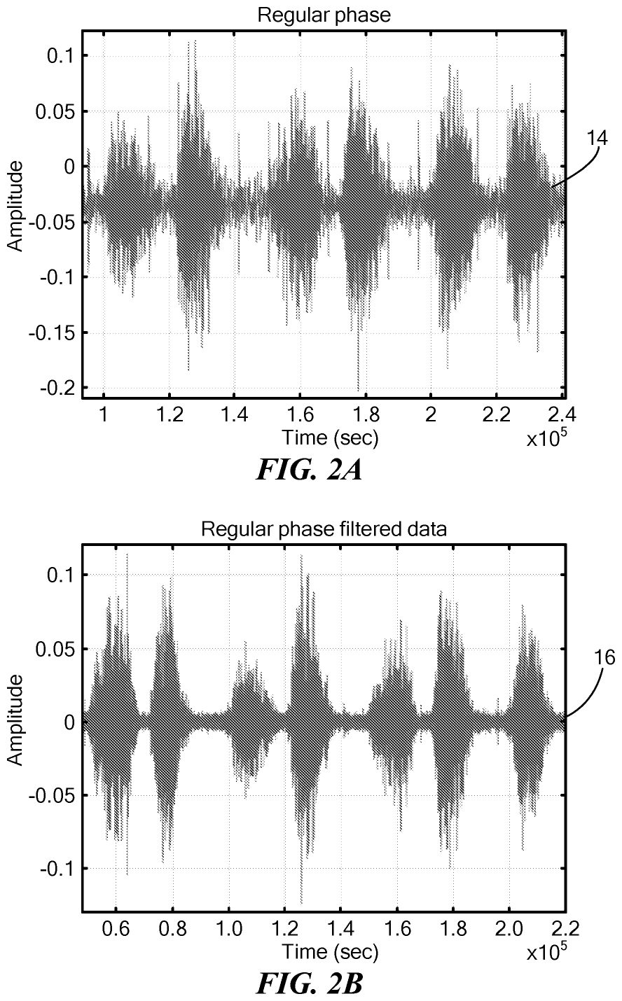

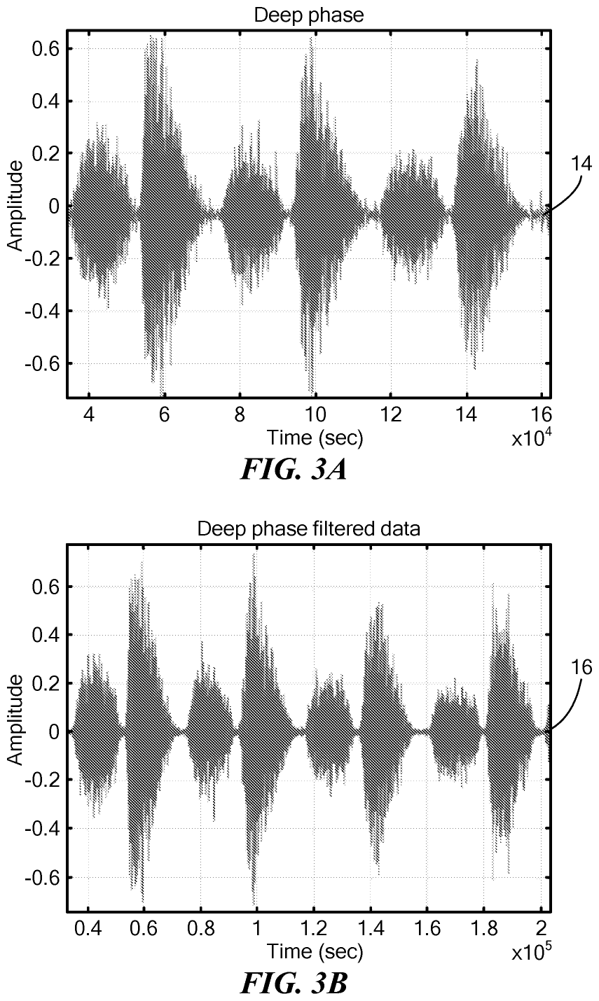

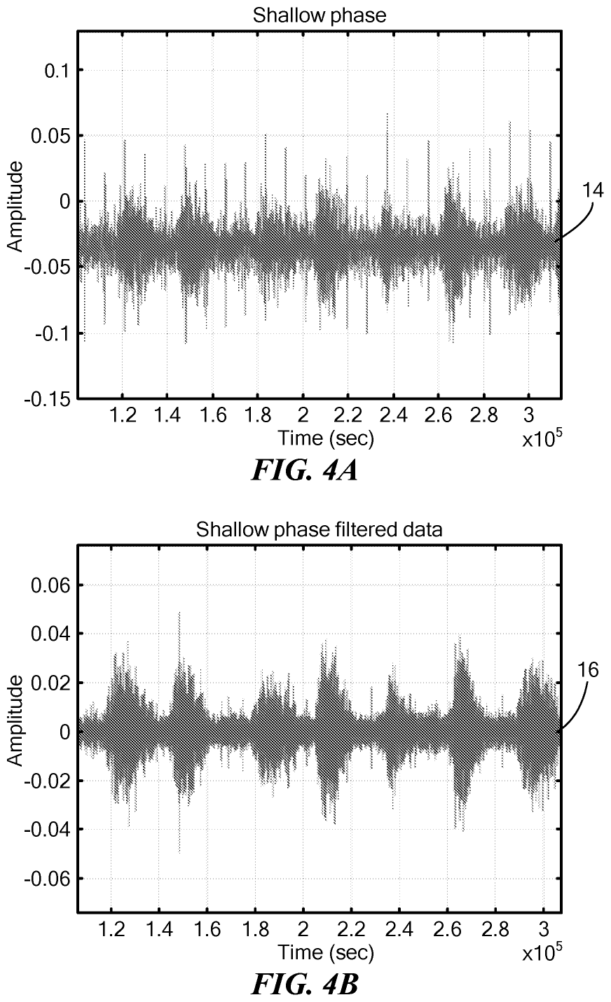

Referring now to FIGS. 1-4, some embodiments include a tracheal acoustic sensor 10 sized and configured to be adhered to the suprasternal notch as depicted and described in U.S. patent application Ser. No. 16/544,033, the entirety of which is incorporated herein by reference. The acoustic sensor 10 is configured to measure sounds emanating from an airflow through the trachea of a mammal, whether human or animal. The acoustic sensor 10 may be in communication with a remote controller 12, for example, wirelessly, the controller 12 having processing circuitry having one or more processors configured to process sound received from the acoustic sensor 10. For example, the controller 12 may be a Smartphone or dedicated control unit having processing circuitry that wirelessly receives an unfiltered sound waveform 14 from the acoustic sensor 10 for further processing. The unfiltered sound waveform 14 may include artifacts such the heartbeat which can obscure the onset and offset of respiratory phases. As used herein, the term respiratory phase refers to inhalation or exhalation, each of which has its own onset and offset times during its respective respiratory phase. FIGS. 2-4 illustrate the sounds amplitude, in decibels of the various respiratory states associated with breathing, namely, shallow, regular, and deep breathing for both the unfiltered sound waveform 14 and a filtered sound waveform 16, as discussed in more detail below.

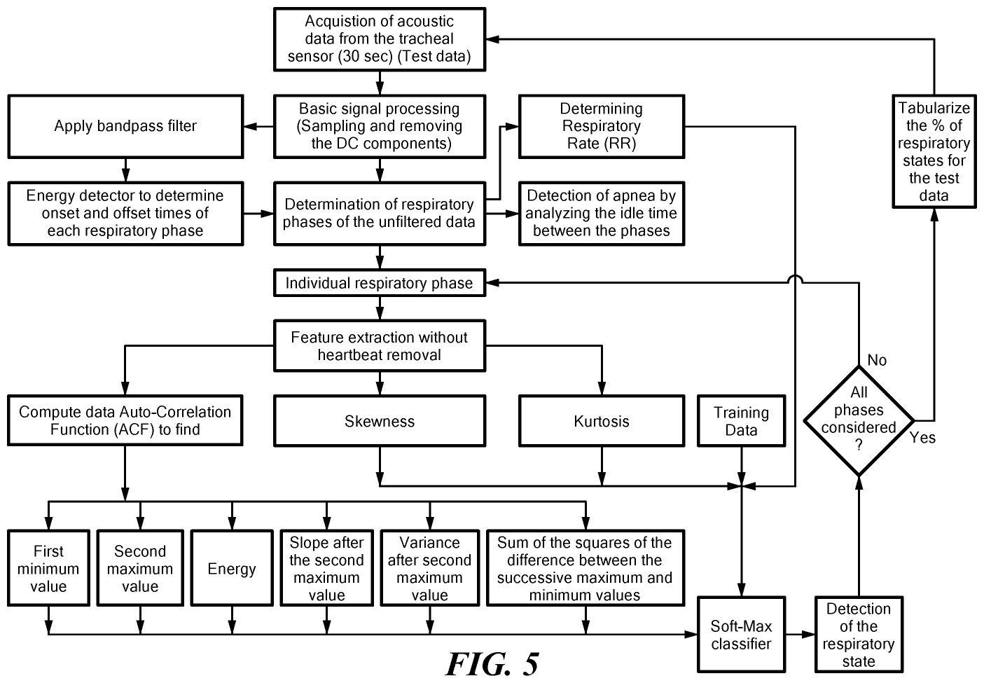

Referring now to FIG. 5, the acoustic sensor 10 may acquire a sound signal for a predetermined time period, for example, continually every thirty seconds, in the form of the unfiltered sound waveform 14 and transmit that signal to the controller 12 for processing. For example, the processing circuitry of controller 12 may be configured to perform basic signal processing on the unfiltered sound waveform 14, in particular, to sample the data and to remove DC components. The processing circuitry may further be configured to apply a bandpass filter to remove sounds associated with the heartbeat and use an energy detector to determine onset and offset times for a respective respiratory phase to create a filtered waveform 16. The respiratory phases may then be determined from the filtered sound waveform 14 and a respiratory rate may be determined as well as whether the mammal has sleep apnea by analysis of the idle times between each respiratory phase. An individual respiratory phase is then isolated and analyzed using first and second order statistics. For example, histograms are computed of the unfiltered sound waveform 14 to provide an estimate of the data probability density function (PDF). In one embodiment, the PDF is obtained using a Gaussian kernel applied to the histogram. Statistical measures are then obtained from the estimated PDF, for example, Entropy, Skewness, and Kurtosis. The Entropy provides a measure of randomness or uncertainty within the PDF, with a maximum uncertainly associated with uniform distribution, i.e. a flat PDF. The Skewness provides information on the degree of asymmetry of the data around its mean. The higher the skewness the more of the data asymmetry. Symmetric data distributions have zero Skewness. The Kurtosis is a measure of whether the data is heavy-tailed or light-tailed relative to a normal distribution and it is used as a measure of outliers in the data.

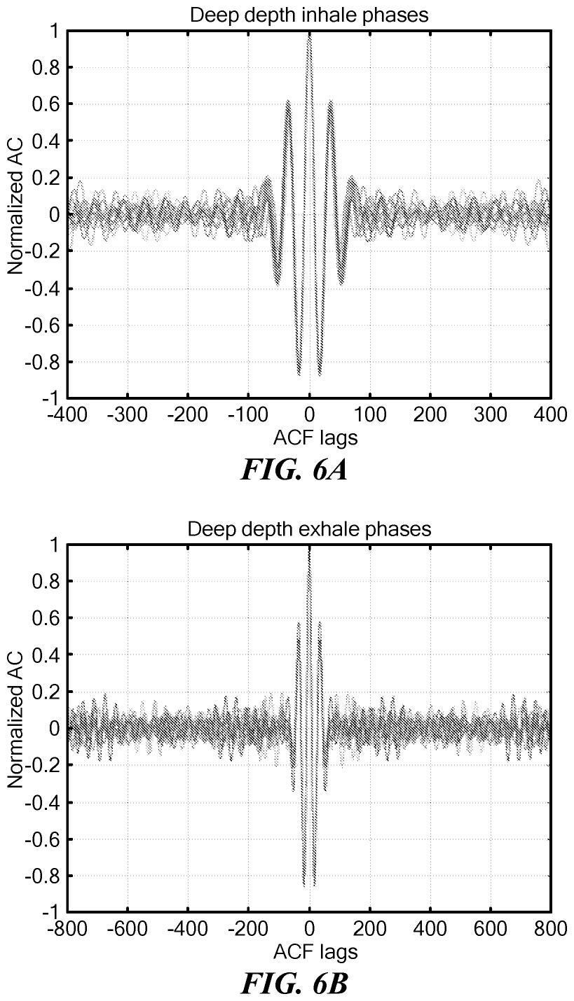

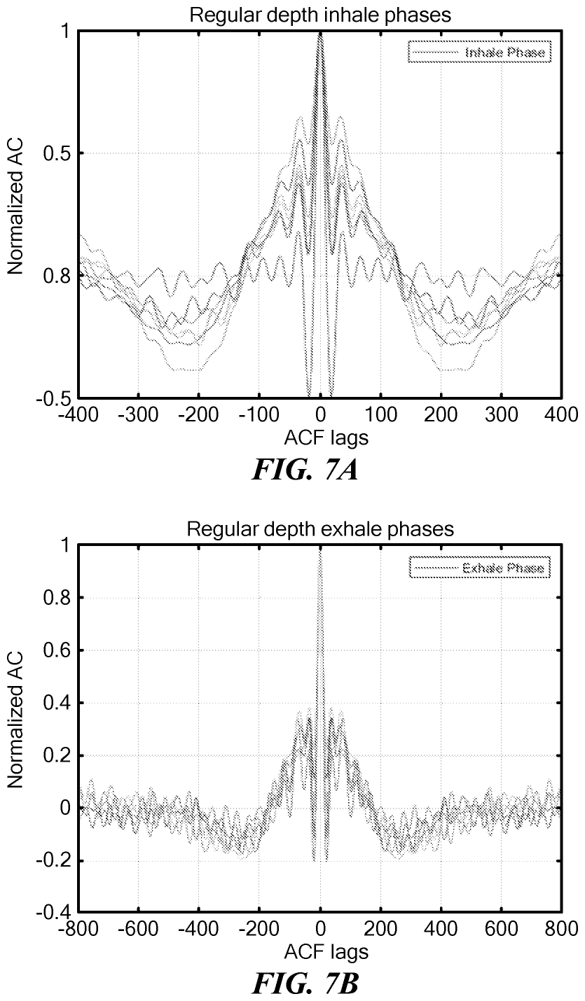

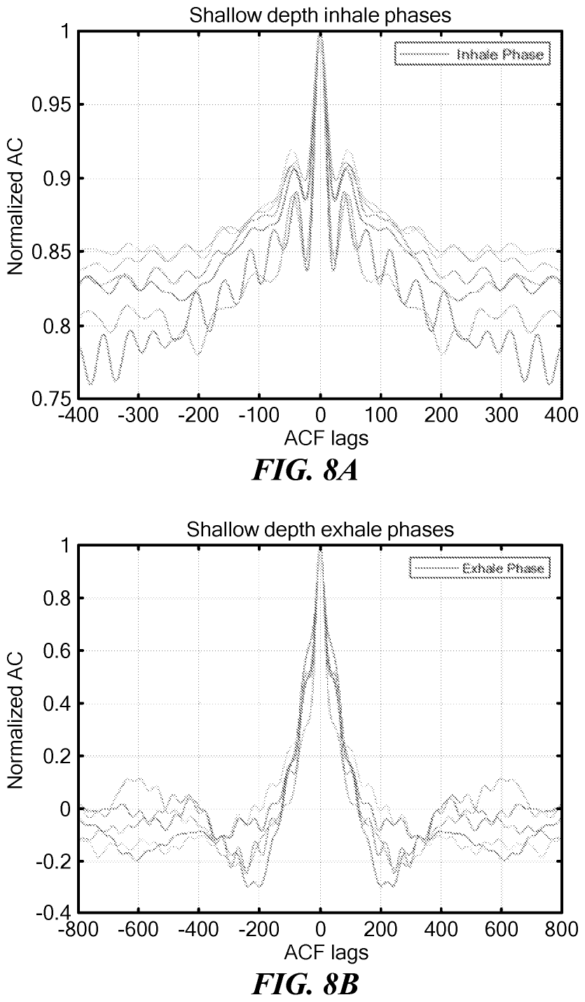

Continuing to refer to FIG. 5, second order statistics on the unfiltered date 14 may further be performed. For example, an estimate of the autocorrelation function (ACF) of the original unfiltered data for each respiratory phase. This estimate is generated using time-averaging of the data lagged product terms. The biased formula of the time-average estimate of ACF may be used and the ACF at the first 1000 lags including the zero lag are analyzed, although any number of lags may be analyzed. The estimated ACF for different time-lags is viewed as a curve plotted or evaluated at 1000 samples. The curve is normalized by its maximum value that occurs at the zero-lag, i.e., first sample of the curve. For example, as shown in FIGS. 6-8, exemplary ACF curves are shown for each respiratory phase for each respiratory state. From the normalized and unnormalized ACF curve, a plurality of features are extracted and include, but are not limited to, (a) the first minimum value of the normalized ACF curve; (b) the second maximum value of the normalized ACF curve; (c) The value of the unnormalized ACF curve at the zero lag; (d) variance after the normalized ACF curve second maximum value; (e) slope after the normalized ACF curve second maximum value; and (f) sum of the squares of the difference between successive normalized ACF curve maximum and minimum. The first minimum value represents the last smallest value of the ACF curve before it rises. With a normalized maximum AFC, to unite value, the feature considered describes the degree of ACF dropping from its maximum to first minimum values. The slope of the curve at the location of this minimum value is zero. The second maximum value represents the “bouncing” behavior of the normalized ACF curve, rising to its first maximum value after encountering the drop, captured by the first minimum value. It is also noted that the slope of the curve at the location of this maximum value is zero. The value of the unnormalized ACF curve at the first sample is equal to the ACF at zero-lag. It also represents the average of the squares of the data values over the respiratory phase considered. The slope after the second maximum value represents a decay in values after the second peak of the ACF curve. The decay behavior is indicated by fitting a straight line to the remaining of the AFC curve, and finding its slope. The line fitting is performed using linear regression. The sum of the squares of the difference between successive maximum and minimum represents the degree of fluctuations, or lack off, of the ACF curve values around its decay line defined by decay behavior. It is computed by finding the ACF curve maxima and minima, after the second peak, and then summing the squares of the differences between every two consecutive maximum-minimum values as well as every two consecutive minimum-maximum values.

Continuing to refer to FIG. 5, a classifier may be applied to at least one of the features from the group (a)-(f) discussed above to determine a percentage of the data from the ACF curve belonging to each respiratory state. In one configuration, all six features are input into the classifier, which may be, for example, a Soft-Max classifier. The classifier may be trained with training data of known sound data during a particular respiratory state. For example, from each subject, data was collected for the three respiratory states; deep, normal, and shallow breathing. The respiratory phases were separated, and the proposed features discussed above were extracted from each phase. The features belonging to all phases of the same respiratory state, and for all three states, are used to train the classifier. In addition to the ACF curve data, the PDF curve data and the determined respirate rate are each input into the classifier. The classifier may then calculate a percentage of each of the determined respiratory states of the plurality of respiratory states during the predetermined period of time based on the classification in the ACF and the PDF curves during the predetermined time period. In particular, for both inhalation and exhalation the classifier calculates a percentage of ACF, PDF, and respiratory rate data that is associated with a particular respiration state, for example, shallow, regular, or deep breathing. The respiratory state having a highest percentage during the predetermined time period is the dominant respiratory state.

It will be appreciated by persons skilled in the art that the present embodiments are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A method of determining respiratory states, comprising:
measuring an unfiltered sound waveform emanating from an airflow through a mammalian trachea for a predetermined time period;
applying time-averages to each of a plurality of respiratory phases of the unfiltered sound waveform to create normalized and unnormalized autocorrelation function (ACF) curves;
determining from the normalized and unnormalized ACF curves at least one feature from a first group of features consisting of:
(a) a first minimum value of the normalized ACF curve;
(b) a second maximum value of the normalized ACF curve;
(c) a value of the unnormalized ACF curve at zero lag;
(d) variance after the normalized ACF curve second maximum value;
(e) slope after the normalized ACF curve second maximum value; and
(f) sum of the squares of the difference between successive normalized ACF curve maximum and minimum values;
applying a classifier to the at least one feature from the group of features; and
determining a respiratory state of a plurality of respiratory states based at least in part on the classification of the at least one features from the first group of features.

2. The method of claim 1, further comprising filtering the unfiltered sound waveform to attenuate sounds emanating from a mammalian heartbeat to create a filtered sound waveform and determining onset and offset times for each of a plurality of respiratory phrases from the filtered sound waveform.

3. The method of claim 2, further comprising determining an individual respiratory phase from the filtered sound waveform to determine in part a respiratory rate.

4. The method of claim 3, wherein applying the classifier further includes applying the classifier to the determined respiratory rate.

5. The method of claim 1, further comprising calculating a percentage of each of the determined respiratory states of the plurality of respiratory states over the predetermined period of time based on the classification, and wherein the determined respiratory state having a highest percentage is a dominant respiratory state.

6. The method of claim 5, wherein the plurality of respiratory states includes deep, normal, and shallow breathing.

7. The method of claim 1, wherein measuring the unfiltered sound waveform emanating from the airflow through the mammalian trachea for the predetermined time period includes measuring the unfiltered sound waveform from an acoustic measurement device positioned on a suprasternal notch of the mammalian trachea.

8. The method of claim 1, further comprising:
computing the histogram of each of the plurality of respiratory phases of the unfiltered sound waveform to create an estimate of the probability density function (PDF).
determining from the PDF curve at least one feature from a second group of features consisting of:
(g) entropy;
(h) skewness; and
(i) kurtosis.

9. The method of claim 1, wherein determining from the ACF curve at least one feature from the first group of features consisting of (a)-(f) includes determining each of features (a)-(f) from the first group of features consisting of (a)-(f).

10. The method of claim 1, wherein the classifier is a Soft-Max classifier.

11. The method of claim 1, wherein the predetermined time period is between 10-30 seconds and the plurality of time lags includes at least 1000 time lags.

12. A system for determining respiratory states, comprising:
an acoustic measuring device sized and configured to be adhered to a suprasternal notch;
a controller in communication with the acoustic measuring device, the controller having processing circuitry configured to:
receive an unfiltered sound waveform from the acoustic device of an airflow through a mammalian trachea for a predetermined time period;
apply time-averages to each of a plurality of respiratory phases of the unfiltered sound waveform to create normalized and unnormalized autocorrelation function (ACF) curves;
determine from the normalized and unnormalized ACF curve at least one feature from a first group of features consisting of:
(a) a first minimum value of the normalized ACF curve;
(b) a second maximum value of the normalized ACF curve;
(c) a value of the unnormalized ACF curve at zero lag;
(d) variance after the normalized ACF curve second maximum value;
(e) slope after the normalized ACF curve second maximum value; and
(f) sum of the squares of the difference between successive normalized ACF curve maximum and minimum values;
apply a classifier to the at least one feature from the first group of features; and
determine a respiratory state of a plurality of respiratory states based at least in part on the classification of the at least one features from the first group of features.

13. The system of claim 12, wherein the processing circuitry is further configured to filter the unfiltered sound waveform to attenuate sounds emanating from a mammalian heartbeat to create a filtered sound waveform and determining onset and offset times for each of a plurality of respiratory phrases from the filtered sound waveform.

14. The system of claim 13, wherein the processing circuitry is further configured to determine an individual respiratory phase from the filtered sound waveform to determine a respiratory rate.

15. The system of claim 14, wherein application of the classifier further includes applying the classifier to the determined respiratory rate.

16. The system of claim 12, wherein the processing circuitry is further configured to calculate a percentage of each of the determined respiratory states of the plurality of respiratory states based on the classification, and wherein the determined respiratory state having a highest percentage is a dominant respiratory state.

17. The system of claim 12, wherein the processing circuitry is further configured to:
compute a histogram of each of the plurality of respiratory phases of the unfiltered sound waveform to create an estimate of the probability density function (PDF); and
determine from the PDF curve at least one feature from a second group of features consisting of:
(g) entropy;
(h) skewness; and
(i) kurtosis.

18. The system of claim 12, wherein the determination from the ACF curve at least one feature from the first group of features consisting of (a)-(f) includes determining each of features (a)-(f) from the first group of features consisting of (a)-(f).

19. The system of claim 12, wherein the classifier is a Soft-Max classifier.

20. A method of determining respiratory states, comprising:
measuring an unfiltered sound waveform emanating from an acoustic measurement device positioned on a suprasternal notch of a mammalian trachea of an airflow through a mammalian trachea for a predetermined time period;
determining an individual respiratory phase from the unfiltered sound waveform to determine a respiratory rate;
applying time-averages to each of a plurality of respiratory phases of the unfiltered sound waveform to create normalized and unnormalized autocorrelation function (ACF) curves;
determining from the normalized and unnormalized ACF curves from a first group of features consisting of:
(a) a first minimum value of the normalized ACF curve;
(b) a second maximum value of the normalized ACF curve;
(c) a value of the unnormalized ACF curve at zero lag;
(d) variance after the normalized ACF curve second maximum value;
(e) slope after the normalized ACF curve second maximum value; and
(f) sum of the squares of the difference between successive normalized ACF curve maximum and minimum values;
compute a histogram of each of the plurality of respiratory phases of the unfiltered sound waveform to create an estimate of the probability density function (PDF); and
determine from the PDF curve at least one feature from a second group of features consisting of:
(g) entropy;
(h) skewness; and
(i) kurtosis;
applying a Soft-Max classifier to the first group of features, the second group of features, and to the determined respiratory rate;
determining a respiratory state of a plurality of respiratory states based at least in part on the applying of the Soft-Max classifier; and
calculating a percentage of each of the determined respiratory states of the plurality of respiratory states during the predetermined period of time based on the classification in the ACF and the PDF curves during the predetermined time period; and
determining a dominant respiratory state, the determined respiratory state having a highest percentage during the predetermined time period is the dominant respiratory state.
